(19) United States
(12) Patent Application Publication
(10) Pub. No.: US 2015/0119782 A1
(43) Pub. Date: Apr. 30, 2015

(54) Buddy Hug

(71) Applicant: Melissa Osiecki, Pittsburgh, PA (US)

(72) Inventor: Melissa Osiecki, Pittsburgh, PA (US)

(21) Appl. No.: 13/968,654

(22) Filed: Oct. 29, 2013

(51) Int. Cl. A61F 5/01

(52) U.S. Cl. CPC A61F 5/0127
USPC 602/30

(57) ABSTRACT

The toe wrap is a new, efficient and fashionably designed toe cast that replaces the traditional method of using tape to stabilize and immobilize a broken toe(s), in a fashionable way, and allow users to accessorize with their attire. The toe wrap wraps around or “hugs” the injured toe and its “buddy toe” (neighboring toe(s)). The interior consists of a soft foam cushion for comfort around the toe(s). The exterior consists of a firm and soft Velcro for stabilization, flexibility and adjust-ability that enables the perfect fit around the toes. The toe wrap is similar to a jewelry piece or decorative accessory, and contains various selections of jewels, glitter, quotes and phrases, patterns and colors, and pictures that enhance the appeal, and allows users of both genders to accessorize and match their attire with various designed toe wraps.

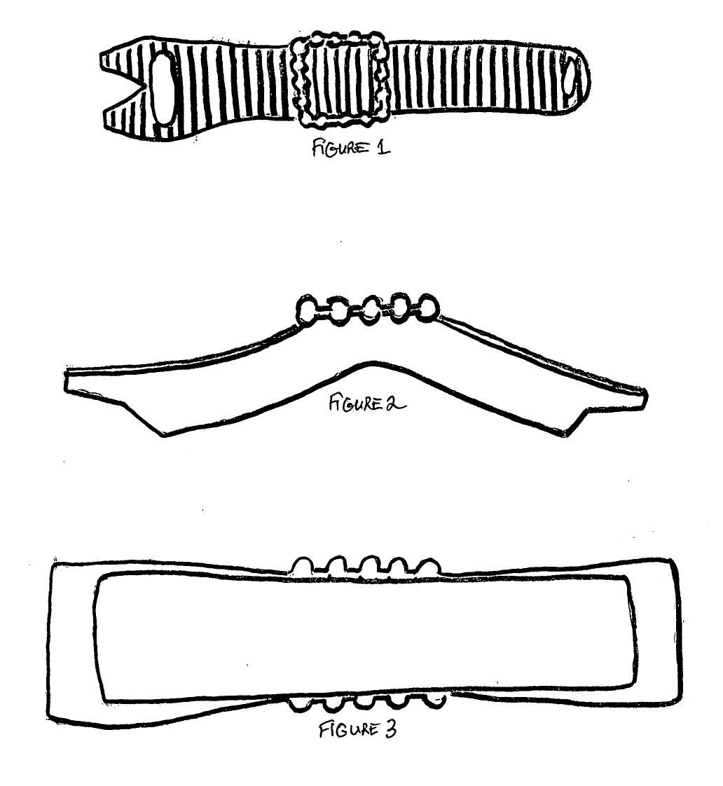

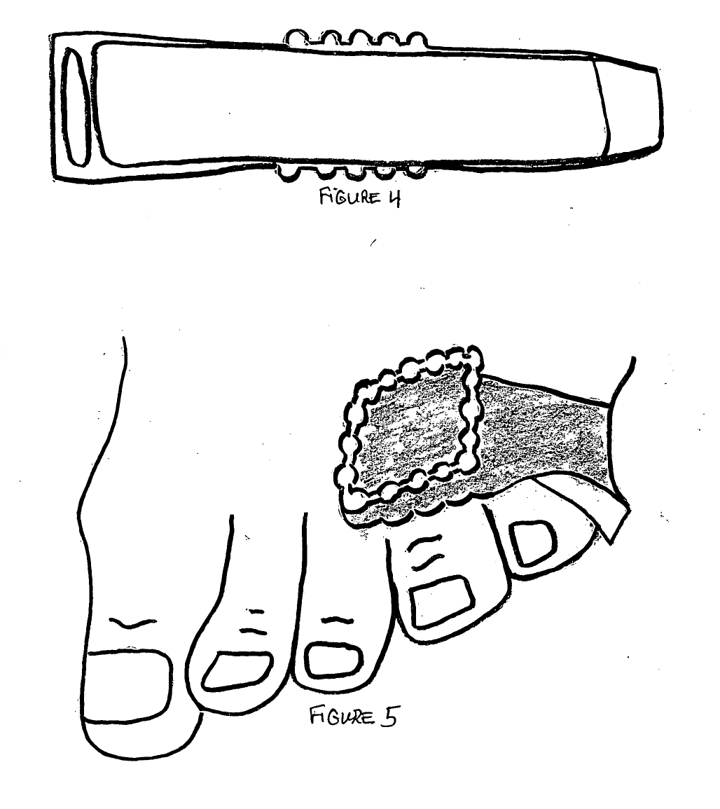

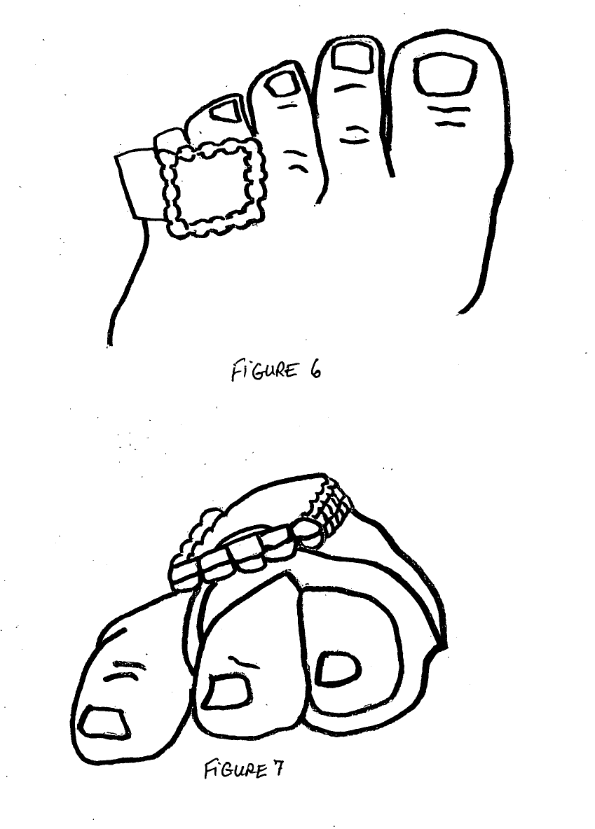

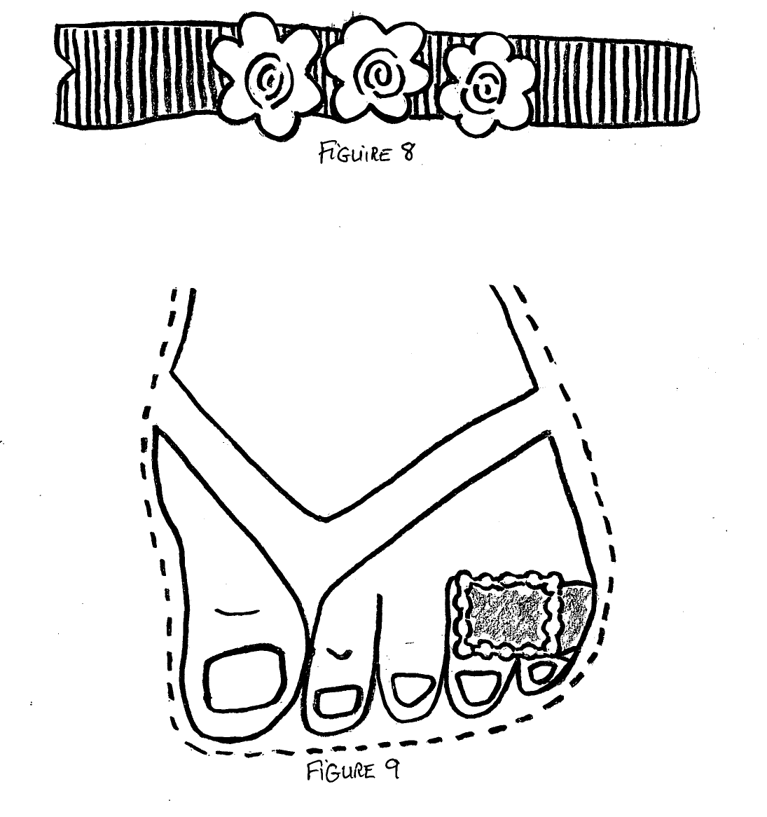

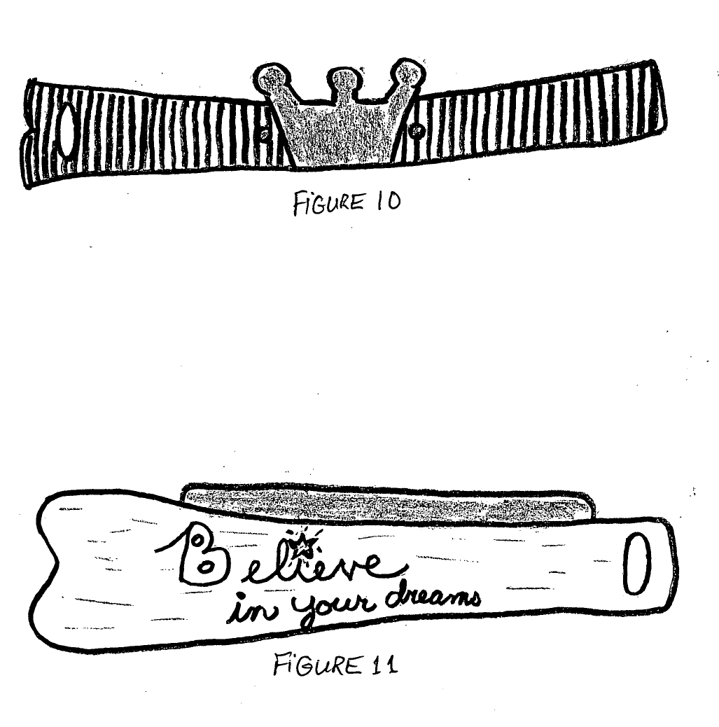

### BACKGROUND

The toe wrap is a stabilizing and fashionably designed wrap that supports, protects, and comforts a broken or injured toe(s) during its healing process. The toe wrap will replace the traditional method of taping broken and injured toes in a more effective and efficient way that is stabilizing, protective, and fashionable. Unlike tape, the toe wrap can be worn numerous times before becoming worn out and therefore, does not require consistent replacement throughout the day. It consists of two layers for extra protection and comforting purposes. The interior of the toe wrap is made of a soft cushion that serves as an extra protective layer and comforts the toe as it heals. The interior wraps entirely around the injured toe and its neighboring uninjured toe, to provide a proper and stabilized position for the injured toe to begin its healing process, just as when wrapped in tape but now wrapped within a firmer, adjustable, and more comfortable and protecting product. The exterior of the toe wrap is made from a soft and flexible material to prevent further irritation and instigated pain, and harm, from rougher materials, and therefore, accompanies the soft and cushion interior with comforting and protecting the toe(s). The toe wrap is designed in various lengths and widths, providing sizes for a more precise fit. It is rectangular shaped, with the top and bottom sides equal in length and width, and the left and right sides equal in length and width. The toe wrap has a Velcro exterior to eliminate buttons, snaps, and hooks, and provide a more comfortable and adjustable closure. The toe wrap is designed in two ways for an additional wrapping method, but has equal effectiveness in both ways. The first design of the toe wrap consists of the top and bottom sides equal in length and width, and the left and right sides equal in length. The user wraps the toe and neighboring toes in the wrap with the closure ending with one of the sides on top of the other. The second design of the toe wrap has the top and bottom sides equal in length but the left and right sides are unequal in length: The right side is more narrow than the left, and can be put through a thin slit-like opening on the right side when wrapping the toes, still ending with the narrow side on top of the wider side in closure.

The exterior of the toe wrap consists of various fashionable designs, patterns and colors, icons and images, beads and jewels, to enhance the appeal and allow users to coordinate with their attire.

### SUMMARY OF THE TOE WRAP

The purpose of the toe wrap is to stabilize, comfort, and protect broken or injured toe(s) during their healing process. The toe wrap wraps firmly and comfortably around the toes, covering all areas of the injured toe(s), and secures the wrapped toe(s) in a proper healing position and refrains the toe(s) from further movement However, with the flexibility and adjustability of the toe wrap, user will be able to move their toes within the wrap, as healing and toe motion progress. It is made of two layers for extra protection: a soft but firm and adjustable exterior, that allows the user to adjust the wrap to a comfortable and precise size around the toe(s), and a soft cushion interior designed for comfort. The exterior is decorated with various patterns, colors, numbers, images and icons, jewels and diamonds, sequins, glitter, and beads, to provide a fashionable appeal.

### DRAWING DESCRIPTIONS

FIG. #1

A top view of the toe wrap. The top view of the toe wrap displays the exterior version of the toe wrap with one end more narrow than the other, and containing a thin opening slit that allows users to put the narrow end through that slit for an additional and optional way of wrapping. In the centre of the exterior is a square jewel that serves as a decorative object.

FIG. #2

A front view of the toe wrap. This picture focuses on the soft cushion interior attached to the exterior, and displays the jewel that is on the center of the exterior.

FIG. #3

A top view of the inside of the toe wrap consisting of equal parallel top and bottom lengths and equal parallel left and right sides. This figure exposes the inner cushion of the toe wrap, and the velcro ends of the inner exterior that allow for adjustability when wrapping the toe(s), and closure after the toe(s) are wrapped.

FIG. #4

Atop view of the interior of the toe wrap that contains equal parallel top and bottom sides in length, but unequal parallel left and right sides in width, containing a thin slit opening on the left side to allow the more narrow right side to go through the opening, as an optional wrap of wrapping the toe wrap.

FIG. #5

A perspective view of the toe wrap being worn around the broken and injured toes, displaying the exterior with a jewel in the center, and displaying the inner cushion protecting and comforting the toes.

FIG. #6

A perspective top view of the toe wrap wrapped around the injured toe and its neighboring toe for stabilization.

FIG. #7

A zoomed-in perspective view of the toe wrap wrapped around the injured toe and its buddy toe.

FIG. #8

One of the exterior designs of the toe wrap.

FIG. #9

A top perspective view of the toe wrap worn with a flip flop.

FIG. #10

A top view of an exterior design of the toe wrap, containing a crown in the center with two jewels on each side of it.

FIG. #11

A front view of a fashionable exterior imaging a decorative phrase, “believe,” for a fashionable appeal.

The following patents have been researched in order to contrast the differences of my invention relating to the toe to past inventions for the toe. The research was performed by a licensed patent attorney and concludes that while various toe ornaments were designed for toe cosmetics and fashion, and invented toe holders, none of their functions were to serve as toe protection, comfort, stabilization and support of broken and injured toes in a fashionable manner.

Toe Pad: Filed Jan. 26, 1938 (U.S. Pat. No. 216,301)

The purpose of the toe pad was only to prevent corns and protect hammertoes. The toe pad is to be worn either directly over the toes or to the rear of the phalangeal joints for the purpose of correcting any deformities or irregularities of the toe. Furthermore, this invention of this toe pad is to improve its function and image of existing toe pads.

The Toe Wrap is designed to support, comfort, and stabilize a broken or injured toe(s) in a fashionably designed toe wrap. The toe wrap wraps entirely around the injured toe and its neighbor toe to provide stabilization to begin the healing process, wrapping in a circular movement around the toe(s), to cover the entire area of injury. The toe wrap consists of two layers for extra comfort and protection purposes. The exterior of the toe wrap is made from soft and flexible material to exclude further instigated pain and harm from rougher materials, and therefore, comforts the toe(s) with its second layer of an interior made from soft cushion.

Toe Holder: Filed May 10, 1971 (U.S. Pat. No. 371,227)

The purpose of the toe holder is to avoid the spreading apart of toes during sun bathing. It's purpose is to refrain toes from spreading apart during sunbathing and avoid exposure to the sun.

My product is to support, comfort, and stabilize broken and injured toes in a fashionably designed toe wrap. The toe wrap adjoins a broken and/or injured toe with an uninjured neighboring toe to provide a stabilized position for the damaged toe for proper healing.

Toe Cap Protector: Filed Jul. 23, 1997 (U.S. Pat. No. 040,484)

The purpose of the toe cap is to protect only the top or tip of the toe and serve merely as a cap. The Toe Wrap wraps around the injured areas of the toe and adjusts to various sizes to fit properly, firmly, and comfortably around the injured toe and neighboring toe.

Two Finger Ring and Two Toe Ring: Filed Aug. 22, 2005 (U.S. Pat. No. 80,913,831)

This product is designed to serve as a cosmetic fashion accessory for the toes, only. It serves no further purpose of stabilizing, cushioning, or protecting broken or injured toes. It focuses more on the design of the ring, rather than serving as a function. It is large enough to hold two fingers or two toes within a ring, and therefore, requiring a larger jewel or stone for decorative purposes. The Two Finger Ring and Two Toe Ring serves as a fashion ring accessory and trend.

The Toe Wrap adjoins an injured toe with its neighboring uninjured toe to provide stabilization, support, and comfort, while the injured toe heals.

Toe Ring Base and Ornamental Toe Ring: Filed Jun. 14, 1976 (U.S. Pat. No. 406,594)

The Toe Ring Base and Ornamental Toe Ring serves as an adjoining toe jewelry accessory. It is a cosmetic and decorative accessory that joins more than one toe together. in a fashionable way but is not designed to support any injured toe(s).

The Toe Wrap is designed to support broken and injured toe(s) with an extra layer of an interior cushion. The Toe Wrap is made of adjustable and flexible material so the user can adjust it to a comfortable and proper precise size around their toe(s).

I claim:

1. A toe wrap for joining one or more injured toes to an adjacent toe for comfort and support while healing, said toe wrap comprising:
a rectangular-shaped strip, made from two layers of material, for wrapping about the injured toe and the adjacent toe before reconnecting to itself and forming a band for comfort, support, and protection; and
means for joining ends of the strip to itself.

2. The toe wrap of claim 1 which consists of four sides: a parallel top and bottom side equal in length, and a parallel left and right side equal in length.

3. The toe wrap of claim 2 wherein the left and right sides each include a hook and loop fastener end for adjustability.

4. The toe wrap of claim 1 which consists of four sides: a parallel top and bottom side equal in length, and a left and right side of different widths, one of the two being wider than the other and having a thin slit opening.

5. The toe wrap of claim 4 wherein the left and right sides each include a hook and loop fastener end for adjustability.

6. The toe wrap of claim 1 wherein one of the left or right sides may be placed into and through the thin slit opening of the other side for an optional way of wrapping about the toes.

7. The toe wrap of claim 1 wherein the rectangular-shaped strip is made of soft and flexible material that allows comfort and adjustability for a precise stable and comfortable size around the toes, and flexibility for eventual movement of toes within the toe wrap as the healing progresses.

8. The toe wrap of claim 1 which includes an interior made from soft foam cushion material for providing an extra layer of protection against further harm to the injured toe.

9. The toe wrap of claim 9 wherein the soft foam cushion interior wraps fully around the injured toe and neighboring toe.

10. The toe wrap of claim 1 which includes an exterior made from soft but firm material with a hook fastener that allows for installation of the toe wrap to a precise, supporting and comforting size around two or more toes.

11. The toe wrap of claim 10 wherein the exterior also serves as a decorative jewelry accessory.

12. The toe wrap of claim 10 wherein the exterior includes a variety of colors and patterns.

13. The toe wrap of claim 10 wherein the exterior includes at least one of: jewels, beads, glitter and sequins.

14. The toe wrap of claim 10 wherein the exterior includes at least one of: images of characters, icons, words and phrases, patterns, sporting objects, numbers, animals, nature, and holiday festivity designs for decoration purposes.
